(19) United States
(12) Patent Application Publication
(10) Pub. No.: US 2014/0165733 A1
(43) Pub. Date: Jun. 19, 2014

(54) METHOD AND DEVICE FOR DETECTING CONFIGURATIONS OF EXTRACORPOREAL BLOOD CIRCUIT, APPARATUS COMPRISING DETECTING DEVICE, AND COMPUTER PROGRAM FOR PERFORMING THE METHOD

(71) Applicants: Olof Jansson, Vellinge (SE); Mattias Holmer, Lund (SE); Anders Nilsson, Sodra Sandby (SE)

(72) Inventors: Olof Jansson, Vellinge (SE); Mattias Holmer, Lund (SE); Anders Nilsson, Sodra Sandby (SE)

(73) Assignee: Gambro Lundia AB, Lund (SE)

(21) Appl. No.: 14/123,397

(22) PCT Filed: May 28, 2012
(86) PCT No.: PCT/EP2012/059929

§ 371 Date: Mar. 7, 2014

Related U.S. Application Data
(60) Provisional application No. 61/491,374, filed on May 31, 2011.

(30) Foreign Application Priority Data
May 31, 2011 (SE) 1150508-8

(51) Int. Cl. A61M 1/30; A61M 1/36

(52) U.S. Cl. CPC A61M 1/301; A61M 1/367
USPC 73/714

(57) ABSTRACT

A method for detecting a property of an extracorporeal line set in an apparatus for extracorporeal blood treatment. The extracorporeal line set is arranged to be connected to a patient. The method includes: measuring a first pressure in a flow path, measuring a second pressure in the flow path, comparing the first and second pressures, and determining a detected extracorporeal line set property based on the pressure comparison.

### TECHNICAL FIELD OF THE INVENTION

The present invention relates to a method for detecting a property of an extracorporeal line set in an apparatus for extracorporeal blood treatment. The invention also relates to a detecting device for detecting a property of an extracorporeal line set in an apparatus for extracorporeal blood treatment. Further, the invention relates to an apparatus for extracorporeal treatment comprising a detecting device, to a computer program for performing the method, and to a computer-readable medium on which such a computer program is stored.

### BACKGROUND ART

Apparatuses for extracorporeal blood treatment, such as hemodialysis, comprise an extracorporeal circuit, through which blood may pass outside the body of a patient to be treated. The extracorporeal circuit comprises a blood access device, through which the apparatus gains access to the circulatory system of the patient. Through the blood access device, blood is withdrawn from the patient, such that it may be treated. After treatment, the blood is returned to the patient through the blood access device. In some apparatus, this blood access device takes the form of two needles, inserted into appropriate blood vessels of the patient to be treated. One retrieves blood from the patient and is referred to as the arterial access. The other returns the treated blood to the patient and is referred to as the venous access. In other apparatuses, the blood access device may be a single needle, which offers both arterial and venous access. Both of these blood access devices may be inserted into a fistula, that has been created surgically by connecting an artery and a vein of the patient. The blood access devices may alternatively be a central venous catheter.

Hemodialysis machines have been developed, which may function in a single needle mode, using a single access device (comprising a single needle or a single catheter), as well as a double needle mode, using a double access device (comprising two needles or two catheters, or one needle or catheter with a double lumen). With such dialysis machines, there is always a risk that the machine is set in a mode that does not correspond to the chosen blood access device. If the machine is set in single needle mode and the blood access device arrangement connected to it is a double blood access arrangement, the time-averaged blood flow rate will be lower than intended and, thus, the dialysis treatment will not be as effective as intended. If, on the other hand, the machine is set in double needle mode, and a single blood access device is connected, there will be no flow into the circulatory system of the patient, but only a recirculation in the tubes connecting the needle to the machine. Hence, there will in fact be no dialysis treatment.

The same problem would generally arise in apparatuses intended for all types of extracorporeal blood treatment, if the apparatus is usable in at least two different modes and if it is possible to connect a blood access device having properties incompatible with the mode set in the machine. The staff handling the apparatus therefore need to be very careful and thoroughly check that the mode chosen and the blood access device match. However, there is always a risk that such a check is omitted.

A similar problem arises when using apparatuses that may be used with blood access devices, particularly needles, of different gauges. Here, the staff in charge of the apparatus may accidentally connect a needle which is of a smaller or larger diameter than what has been prescribed.

Further, problems of the same type may arise with apparatuses that can be connected to the patient using different blood tubing sets.

Thus, there exists a need for a method and device that makes it possible to automatically detect a property of the extracorporeal line set used in an apparatus for extracorporeal blood treatment, such that a mismatch between the operational mode in which the apparatus is set and the actual extracorporeal line set that is connected to the apparatus can be avoided. It is also desirable to be able to automatically detect a property of the extracorporeal line set used in an apparatus for extracorporeal blood treatment, such that a mismatch between a prescribed property and a property of the extracorporeal line set actually connected can be avoided.

### SUMMARY OF THE INVENTION

An object of the invention is to solve or at least lessen the problems mentioned above.

A particular object of the invention is to provide a method for detecting a property of an extracorporeal line set in an apparatus for extracorporeal blood treatment.

Another object of the invention is to provide an apparatus for extracorporeal blood treatment which makes it possible to automatically detect a property of an extracorporeal line set used in the apparatus.

These objects are achieved, in full or at least in part, by a method as claimed in claim 1, with preferred variants defined in the dependent claims.

These objects are also achieved, in full or at least in part, through a detection device as claimed in claim 10, with preferred embodiments defined in the dependent claims.

These objects are also achieved, in full or at least in part, through an apparatus as claimed in claim 19, a computer program as claimed in claim 20, and a computer-readable medium as claimed in claim 21.

According to the invention, a method for detecting a property of an extracorporeal line set in an apparatus for extracorporeal blood treatment, said blood access device being arranged to be connected to a patient, comprises the steps of:

measuring a first pressure in a flow path in said apparatus,

measuring a second pressure in said flow path in said apparatus,

comparing said first and second pressures, and

determining a detected extracorporeal line set property of said extracorporeal line set based on said pressure comparison. This method makes it possible to automatically detect a property of the extracorporeal line set. This, in turn, makes it possible to correct any mismatch of the property of the extracorporeal line set. Determining if said blood access device comprises a single access or double accesses is useful in an apparatus which can be operated in both a single access mode and a double access mode, particularly a single needle mode and a double needle mode. The measurements of the first and second pressures may be understood as discrete measurements or as start and end points of a continuous measurement. As used herein, the term “extracorporeal line set” includes blood access devices and blood tubing sets. Therefore, the property of the extracorporeal line set is at least one of a configuration of a blood access device in said extracorporeal line set, said configuration being a single access or double accesses, and a blood tubing set property of a blood tubing set in said extracorporeal line set.

The pressure comparison may be to calculate a difference between said first and second pressures.

In a variant, the first pressure is measured in a first portion of said flow path, and the second pressure is measured in a second portion of said flow path, said second portion being different from said first portion. In this manner, it is easy to make the pressure comparison in an apparatus having two pressure sensors.

The first portion of the flow path may be connectable to a venous access of a patient who is to undergo the extracorporeal blood treatment, and the second portion of the flow path may be connectable to an arterial access of the patient.

In a variant, the first pressure is measured at a first point of time and the second pressure is measured at a second point of time, said second point of time being different from said first point of time. In this way, the two pressures may be measured using only one pressure sensor. The first and second pressures may be measured at different flow rates, one of which may be zero. It is also possible to measure the first pressure during flow in one direction and the second pressure during flow in the opposite direction.

In a variant, the step of determining a blood access device property may further comprise determining a needle gauge of a needle in said blood access device. The needle gauge is another property which may be prescribed for the treatment to be performed with the apparatus.

Determining a detected blood tubing set property may comprise determining if the blood tubing set is a single needle single pump set, a single needle double pump set, or a double needle set. This is useful if there are several blood tubing sets that are possible to connect to the apparatus for extracorporeal blood treatment.

The method may further comprise the step of measuring a third pressure, wherein

the blood tubing set is determined to be a single needle single pump set if the first pressure is equal to the second pressure, and the third pressure increases,

the blood tubing set is determined to be a single needle double pump set if the first pressure initially decreases and when the first pressure has stabilised the first pressure is equal to the second pressure, and

the blood tubing set is determined to be a double needle set if the first pressure is not equal to the second pressure, and the third pressure increases. The third pressure may be measured using the same pressure sensor as for measuring one or both of the first and second pressures, or using a separate pressure sensor.

In a variant, the method may further comprise the steps of:

setting a predetermined extracorporeal line set property,

comparing said detected extracorporeal line set property with said predetermined extracorporeal line set property, and

if said detected extracorporeal line set property is not equal to said predetermined extracorporeal line set property, generating an alarm event. This makes it possible to improve the safety of the patient and the reliability of the apparatus, since staff in charge of the apparatus may be alerted of possible errors.

The step of generating an alarm event may comprise at least one of displaying an error message, emitting an alarm sound, and switching the apparatus for corporeal blood treatment to an operation mode consistent with said detected blood access device property.

In a variant, the method may further comprise the step of storing the detected extracorporeal line set property. This allows safe journal-keeping.

The apparatus for extracorporeal blood treatment may be a dialysis machine arranged for use in a single needle mode and a double needle mode.

According to the invention, a detection device for detecting a property of an extracorporeal line set in an apparatus for extracorporeal blood treatment comprises a pressure sensing arrangement and a calculation unit, wherein said pressure sensing arrangement is arranged to measure a first pressure in a flow path in said apparatus and a second pressure in the flow path in said apparatus, and wherein said calculation unit is arranged to compare said first and second pressures and to determine a detected extracorporeal line set property of said extracorporeal line set based on said comparison. The detection device of the invention has the same advantages as the inventive method and may, generally be embodied in the same ways as the inventive method. As already mentioned, as used herein, the term “extracorporeal line set” includes blood access devices and blood tubing sets. Therefore, the property of the extracorporeal line set is at least one of a configuration of a blood access device in said extracorporeal line set, said configuration being a single access or double accesses, and a blood tubing set property of a blood tubing set in said extracorporeal line set.

An inventive apparatus for extracorporeal blood treatment comprises a detection device according to the invention.

According to the invention, a computer program comprises software instructions that, when executed on a computer, perform the method of the invention.

On a computer-readable medium according to the invention, a computer program according to the invention is stored.

Other objectives, features and advantages of the present invention will appear from the following detailed disclosure, from the attached claims, as well as from the drawings. It is noted that the invention relates to all possible combinations of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of said element, device, component, means, step, etc., unless explicitly stated otherwise. The steps of any method disclosed herein do not have to performed in the exact order disclosed, unless explicitly stated.

As used herein, the term “extracorporeal circuit” refers to the parts of the apparatus for extracorporeal blood treatment through which blood passes outside the body of the patient during treatment. More specifically, the extracorporeal circuit comprises an extracorporeal line set, blood pumps and equipment for performing the actual treatment of the blood. As an example, in a hemodialysis machine, such equipment may be a membrane. The extracorporeal circuit may, optionally, comprise additional parts, such as compliance vessels.

Further, the term “blood access device” refers to a needle arrangement or catheter arrangement or any similar arrangement that establishes access to the circulatory system of a patient who is to undergo an extracorporeal blood treatment. More particularly, such a needle arrangement may be a single needle or double needles. Generally, a needle may be any vascular access needle.

### BRIEF DESCRIPTION OF THE DRAWINGS

The invention will be described in more detail with reference to the appended schematic drawings, which show an example of a presently preferred embodiment of the invention.

FIG. 1 is a principal diagram showing the layout of an apparatus according to an embodiment of the invention with double needles connected.

FIG. 2 is a principal diagram showing the layout of the apparatus of FIG. 1, but with a single needle connected.

FIG. 3 is a principal diagram showing the layout of the apparatus of FIG. 1, but with single needle double pump.

### DETAILED DESCRIPTION OF PREFERRED EMBODIMENTS OF THE INVENTION

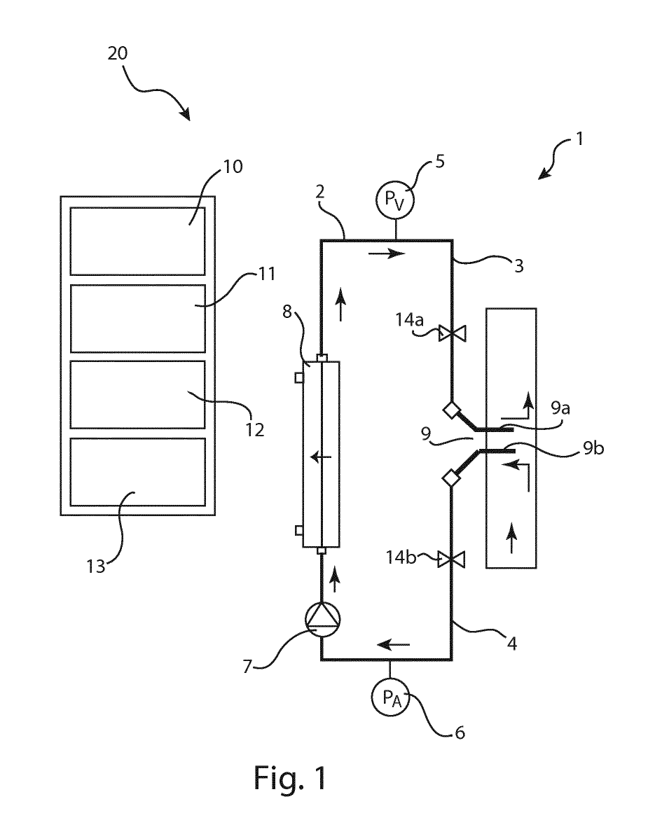

In FIG. 1, an apparatus for extracorporeal blood treatment, here a part of a hemodialysis machine, is shown, denoted generally with the reference numeral 1. The dialysis machine 1 has a flow path 2 made up of flexible tubes through which blood from a patient is to travel to be treated, thus forming an extracorporeal blood circuit. A first portion 3 of the flow path 2 is connectable to a venous access of the patient and a second portion 4 of the flow path 2 is connectable to an arterial access of the patient. The dialysis machine 1 includes a detecting device 20. This detecting device 20 comprises a first pressure sensor 5 connected to the first portion 3 of the flow path 2 for measuring a first pressure in the first portion 3 of the flow path 2, and a second pressure sensor 6 connected to the second portion 4 of the flow path 2 for measuring a second pressure in the second portion 4 of the flow path 2. Further, the dialysis machine 1 comprises a pump 7 for pumping blood through the flow path 2, and a membrane arrangement 8 through which blood may exchange low-molecular compounds with a dialysis fluid in order to remove impurities from the blood. The flow path 2 of the dialysis machine 1 is connectable to the circulatory system of the patient through a blood access device 9, here in the form of a single needle or double needles. Additionally, the detecting device 20 of the dialysis machine 1 has a calculation unit 10, a sound emitter 11, a display 12, and a memory 13. The blood access device 9, the flow path 2, the pump 7, and the membrane 8 form an extracorporeal circuit of the dialysis machine 1.

In the configuration shown in FIG. 1, the flow path 2 of the dialysis machine 1 is connected to blood vessels of the patient using double needles. A first needle 9a connects the first portion 3 of the flow path 2 to a venous access of the patient. A second needle 9b connects the second portion 4 of the flow path to an arterial access of the patient. This configuration is referred to as a double needle configuration.

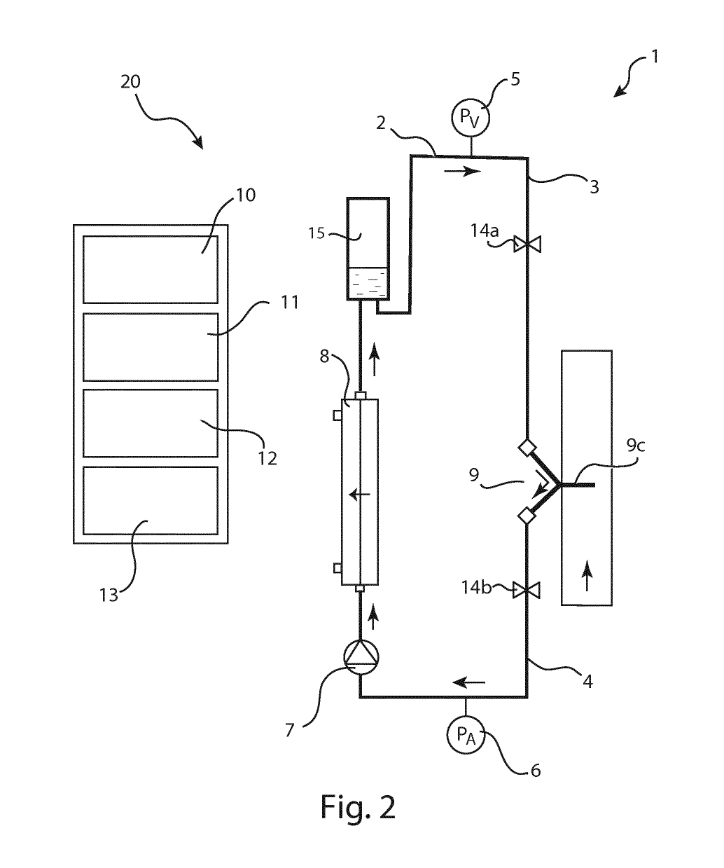

In the configuration shown in FIG. 2, the flow path 2 of the dialysis machine 1 is connected to a blood vessel of the patient using a single needle 9c. The single needle 9c connects the first portion 3 of the flow path 2 and the second portion 4 of the flow path 2 to a blood vessel of the patient. This configuration is referred to as a single needle configuration.

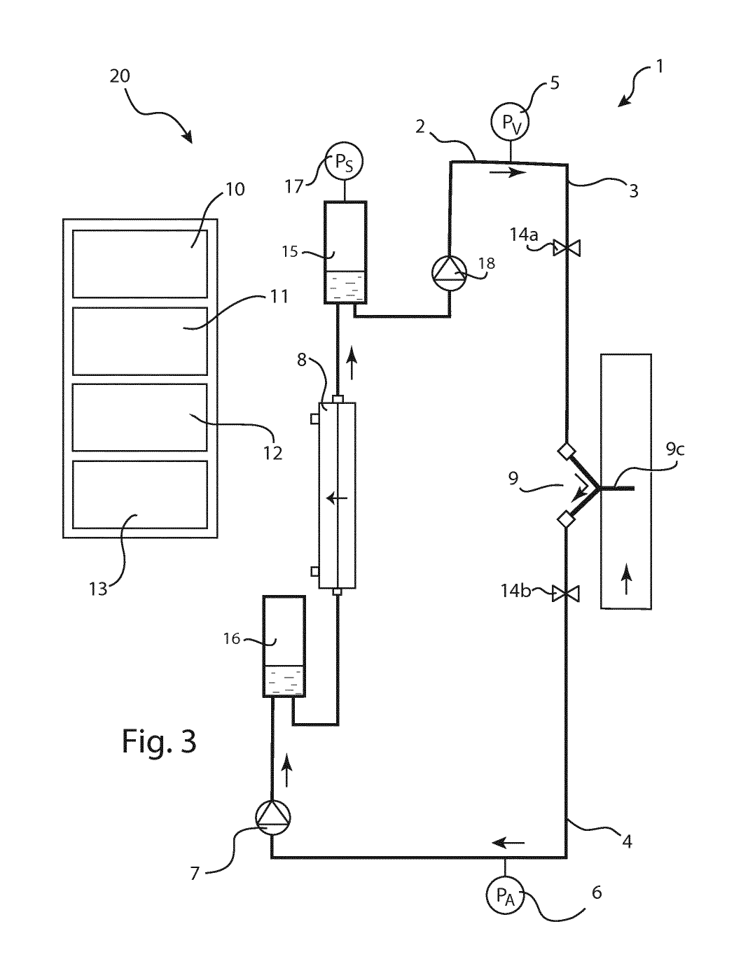

In the single needle configuration, the dialysis machine 1 may comprise clamps 14a, 14b adjacent the single needle 9c. A venous clamp 14a is arranged at the first portion 3 of the flow path 2, and an arterial clamp 14b arranged at the second portion 4 of the flow path 2. During operation of the dialysis machine 1, first, during an arterial pumping cycle, the venous clamp 14a is closed, while the arterial clamp 14b is open, whereby blood is pumped from the patient to the membrane arrangement 8. After a predetermined time, the arterial clamp 14b is closed and the venous clamp 14a is opened, thereby allowing a venous pumping cycle during which treated blood is returned to the patient. Alternatively, instead of using clamps, a second pump (18 in FIG. 3) may be used in addition to the pump 7 shown in FIG. 2. The pump 7 may then be used for the arterial pumping cycle, whereas the second pump may be used for the venous pumping cycle. A first expansion chamber 15 is connected in the flow path 2 as a compliance vessel for building up pressure in the flow path.

In FIG. 3, a double pump setup of a single needle configuration is shown. Here, a second pump 18 is connected after the membrane 8. Further, a second expansion chamber 16 is connected after the second pump 18. A third pressure sensor 17 arranged to measure a system pressure is connected to the second expansion chamber.

The dialysis machine 1 is operable in a single needle mode as well as a double needle mode. In the single needle mode, the blood access device 9 is to be of the single needle configuration, i.e. using the single needle 9c. In the double needle mode, on the other hand, the blood access device 9 is to be of the double needle configuration, i.e. using the two needles 9a, 9b.

A method for automatically detecting the configuration of the blood access device 9 will now be described. This may be referred to as a first test. In general, a physician will have made a prescription for the treatment of a specific patient. This prescription is entered into a controller of the dialysis machine 1 and may include, e.g., the specific composition of the dialysis fluid to be used, as well as an instruction regarding which operational mode to use, i.e. single needle mode or double needle mode. Thus, a prescribed configuration of the blood access device 9 is set in the controller of the dialysis machine 1. At some point of the start-up phase, the extracorporeal flow path 2 may be filled with either a specific priming solution or blood from the patient. During said start-up phase, a first pressure P_{V} is measured by the first pressure sensor 5. The first pressure sensor 5 is normally referred to as a venous pressure sensor, and the first pressure P_{V} may therefore be referred to as a venous pressure. During start-up, a second pressure P_{A} is also measured, using the second pressure sensor 6. The second pressure sensor 6 is normally referred to as an arterial pressure sensor, and the second pressure P_{A} may therefore be referred to as an arterial pressure. The pressures P_{V} and P_{A} may be static or dynamic. It should here be noted that, the pressures should be measured with the clamps open, in order to avoid risking bursting the tubes if an incorrect set of tubes has been connected. The pressures will generally be measured with blood or priming solution flowing in the flow path 2.

In the calculation unit 10, possibly included in the controller of the dialysis machine 1, a pressure drop or pressure difference ΔP is calculated as a difference between the first pressure P_{V} and the second pressure P_{A}. The pressures used for the calculation may be mean values of a number of pressure measurements or filtered pressure measurements, in order to compensate for noise generated by pressure pulsations from the blood pumps or from the heart. This pressure difference ΔP is compared to a reference value for the pressure difference, which may have been empirically determined for each operational mode of the dialysis machine 1. If the dialysis machine 1 is set in one of single needle mode and double needle mode, and the corresponding configuration of the blood access device is used, the calculated pressure difference ΔP will be essentially equal to the reference value, within limits of tolerance. If, however, the dialysis machine 1 is set in single needle mode, and the venous and arterial clamps are open, and double needles 9a, 9b are used, the flow resistance through the flow path will be higher than intended, and therefore the calculated pressure difference ΔP will be higher than the reference value. Similarly, if the dialysis machine 1 is set in double needle mode and a single needle 9c is used, there will be almost no flow through the needle 9c, but mainly recirculation in the flow path 2. Therefore, the pressure drop ΔP will be lower than the reference value. In either case, if the wrong needle configuration is used with the operational mode set in the dialysis machine, an alarm event is generated. An alarm sound is emitted by the sound emitter 11, alerting the staff in charge of the dialysis machine 1 of the error. An error message is displayed on the display 12, indicating the nature of the error, for instance “Single needle mode set, double needle detected”. The display 12 may also display a question to the staff, asking if the mode of the dialysis machine should be switched. Thus, if the staff deems it more appropriate to switch the mode of the dialysis machine 1 than to replace the blood access device used with another of the correct configuration, the dialysis machine 1 may switch from one mode to another. Every time an incorrect combination of operation mode of the dialysis machine 1 and configuration of the connected blood access device 9 is detected, the detected configuration of the blood access device may be stored in the memory 13 for journal-keeping. For improved security, the detected configuration may be stored each time the dialysis machine is started, even when a correct combination of operation mode and needle configuration is detected.

In order to determine what type of blood tubing set 2 is connected to the dialysis machine 1 a second test may be performed in addition to the first test described above. Here, a third pressure is measured using the first pressure sensor 5. This third pressure is measured with the venous clamp 14a closed.

If the third pressure increases rapidly when the pump 7 is running for a short while, this indicates that there is no expansion chamber between the membrane 8 and the pressure sensor 5, implying that the blood tubing set is a double needle set, such as in FIG. 1. The pump 7 may for instance run slowly until a certain pressure level is reached and the time needed is then used for determining the blood tubing set. An alternative way is to let the pump 7 add a known small volume and to use the pressure measured after the volume has been added to determine the blood tubing set.

If the third pressure increases slowly, this indicates that the first expansion chamber 15 is filled to a normal level for single needle mode, since air present in the first expansion chamber 15 may be compressed. Thus, the slowly increasing third pressure implies that the blood tubing set is a single needle set, such as in FIG. 2.

It may in this context be noted that if the first expansion chamber 15 is completely full, there will be a rapid increase of the third pressure, thus falsely indicating that the blood tubing set is a double needle set. However, if the dialysis machine 1 is set in double needle mode this does not pose any problem and dialysis may safely be continued. However, if the dialysis machine 1 is set in single needle mode, the level in the first expansion chamber 15 will have to be adjusted. Possibly, the blood tubing set 2 could be exchanged instead, but this would generally be less efficient.

With reference to FIG. 3, the second test is not necessary to perform for determining the type of set attached. This is due to that the third pressure sensor 17 is giving the information already during the first test. During the first test, the pressure measured by the third sensor is increasing and thereby indicating that a single needle double pump set is attached. The second test may be used as an addition to the first test or as a separate test. As discussed above, the first test is mainly used for determining a property of the blood access device 9, whereas the second test is used for determining what type of blood tubing set is connected. In the same way as described for the first test, an alarm event may be generated if an incorrect blood tubing set is detected, and the result of the test be stored for journal keeping.

In Table 1 a number of scenarios that may be encountered are given, by way of example. Table 2 lists explanations of notations used in Table 1.

TABLE 1

| | | | | Pressures | | | | |
|---|---|---|---|---|---|---|---|---|
| | | | | | Second | | | |
| | | | | First | test, | | | |
| | | | | test, | venous | | Test | |
| | | | Needle | clamps | clamp | Test | out- | |
| Set | SP/DP | Mode | Config. | open | closed | indication | come | Action |
| SN | SP | SN | SN | Pᵥ = P_{A} | Pᵥ | SN | OK | Run |
| | | | | | increases | config. | | SNSP |
| | | | | | slowly | SP | | |
| | | | | | | SN set | | |
| | | | | | Pᵥ | SN | Check- | Issue |
| | | | | | increases | config. | up | alarm - |
| | | | | | rapidly | SP | needed | Check |
| | | | | | | Unclear | | setup |
| | | | | | | set | | |
| DN | SP | DN | DN | Pᵥ≠P_{A} | Pᵥ | DN | OK | Run DN |
| | | | | | increases | config. | | |
| | | | | | rapidly | SP | | |
| | | | | | | DN set | | |
| SN | DP | SN | SN | Pᵥ | Test | SN | OK | Run |
| | | | | first | not | config. | | SNDP |
| | | | | falls, | needed, | DP | | |
| | | | | then | but Pₛ | SNDP | | |
| | | | | constant | increases | set | | |
| | | | | Pᵥ = P_{A} | and | | | |
| | | | | Pₛ | Pᵥ is | | | |
| | | | | rises | un- | | | |
| | | | | | changed | | | |
| SN | SP | DN | SN | Pᵥ = P_{A} | Pᵥ | SN | Mode | Switch |
| | | | | | increases | config. | and | to SNSP |
| | | | | | slowly | SP | set do | mode or |
| | | | | | | SN set | not | issue |
| | | | | | | | match | alarm |
| | | | | | Pᵥ | SN | Check- | Issue |
| | | | | | increases | config. | up | alarm - |
| | | | | | rapidly | SP | needed | Check |
| | | | | | | Unclear | | setup |
| | | | | | | set | | |
| DN | SP | SN | DN | Pᵥ≠P_{A} | Pᵥ | DN | Mode | Switch |
| | | | | | increases | config. | and | to DN |
| | | | | | rapidly | SP | set do | mode or |
| | | | | | | DN set | not | issue |
| | | | | | | | match | alarm |
| SN | DP | DN | SN | Pᵥ | Test | SN | Mode | Switch |
| | | | | first | not | config. | and | to SNDP |
| | | | | falls, | needed, | DP | set do | mode or |
| | | | | then | but Pₛ | SNDP | not | issue |
| | | | | constant | increases | set | match | alarm |
| | | | | Pᵥ = P_{A} | and | | | |
| | | | | Pₛ | Pᵥ is | | | |
| | | | | rises | un- | | | |
| | | | | | changed | | | |
| SN | SP | SN | DN | Pᵥ≠P_{A} | Pᵥ | DN | Set | Switch |
| | | | | | increases | config. | and | to DN |
| | | | | | slowly | SP | needle | mode or |
| | | | | | | SN set | config. | issue |
| | | | | | | | do | alarm |
| | | | | | | | not | |
| | | | | | | | match | |
| | | | | | Pᵥ | DN | Check- | Issue |
| | | | | | increases | config. | up | alarm - |
| | | | | | rapidly | SP | needed | Check |
| | | | | | | Unclear | | setup |
| | | | | | | set | | |
| DN | SP | DN | SN | Pᵥ = P_{A} | Pᵥ | SN | Set | Issue |
| | | | | | increases | config. | and | alarm - |
| | | | | | rapidly | SP | needle | add |
| | | | | | | DN set | config. | expansion |
| | | | | | | | do | chamber |
| | | | | | | | not | or |
| | | | | | | | match | needle |
| SN | DP | SN | DN | Pᵥ | Test | DN | Set | Issue |
| | | | | constant | not | config. | and | alarm |
| | | | | Pᵥ≠P_{A} | needed, | DP | needle | |
| | | | | Pₛ | but Pₛ | SNDP | config. | |
| | | | | rises | increases | set | does | |
| | | | | | and | | not | |
| | | | | | Pᵥ is | | match | |
| | | | | | un- | | | |
| | | | | | changed | | | |
| SN | SP | DN | DN | Pᵥ≠P_{A} | Pᵥ | DN | Set | Switch |
| | | | | | increases | config. | does | to DN |
| | | | | | slowly | SP | not | mode or |
| | | | | | | SN set | match | issue |
| | | | | | | | with | alarm |
| | | | | | | | mode | |
| | | | | | | | and | |
| | | | | | | | needle | |
| | | | | | | | config. | |
| | | | | | Pᵥ | DN | Check- | Issue |
| | | | | | increases | config. | up | alarm - |
| | | | | | rapidly | SP | needed | Check |
| | | | | | | Unclear | | setup |
| | | | | | | set | | |
| DN | SP | SN | SN | Pᵥ = P_{A} | Pᵥ | SN | Set | Issue |
| | | | | | increases | config. | does | alarm - |
| | | | | | rapidly | SP | not | add |
| | | | | | | DN set | match | expansion |
| | | | | | | | with | chamber |
| | | | | | | | mode | or |
| | | | | | | | and | needle |
| | | | | | | | needle | |
| | | | | | | | config. | |
| SN | DP | DN | DN | Pᵥ | Test | DN | Set | Issue |
| | | | | constant | not | config. | does | alarm |
| | | | | Pᵥ≠P_{A} | needed, | DP | not | |
| | | | | Pᵥ | but Pₛ | SNDP | match | |
| | | | | rises | increases | set | with | |
| | | | | | and | | and | |
| | | | | | Pᵥ is | | needle | |
| | | | | | un- | | config. | |
| | | | | | changed | | | |

TABLE 2

| Column | Short form | Full form |
|---|---|---|
| 1 | Set | Blood tubing set in use |
| 2 | SP/DP | Blood pumps in use (Single or |
| | | Double Pump, i.e. SP or DP) |
| 3 | Mode | The mode of the dialysis machine |
| | | control system, selected by the |
| | | operator to either SN or DN |
| 4 | Needle config. | Needle configuration |
| 5 | Pressures | Result of pressure test |
| 6 | Test indication | Test indication of needle |
| | | configuration, SP or DP and blood |
| | | tubing set in use |
| 7 | Test outcome | Test outcome: blood tubing set, |
| | | machine mode and needle |
| | | configuration agree or not |
| 8 | Action | Action taken by the machine or |
| | | proposed action for the operator |

The prescription entered into the controller of the dialysis machine 1 may also include the recommended needle gauge, i.e. needle diameter. If the needle or needles actually connected to the flow path 2 of the dialysis machine are of a thinner or thicker diameter than the recommended needle gauge, the calculated pressure difference ΔP will deviate from a reference value determined for the recommended needle gauge. In such case, an alarm event may be generated in the same way as for an incorrect combination of operation mode and needle configuration. If the needle actually used is of a smaller inner diameter than the prescribed needle gauge, the pressure drop over the needle will be greater than the reference value for the prescribed needle gauge. Analogously, if the needle actually used is of a larger inner diameter than the prescribed needle gauge, the pressure drop over the needle will be smaller than the reference value for the intended needle gauge.

If the dialysis machine 1 is of a single needle configuration using clamps, such as shown in FIG. 2, the needle gauge may be checked by measuring pressure with the arterial clamp 14b open and using the arterial pressure sensor 6. The first pressure should in that case be measured at a first flow rate and the second pressure should be measured at a second flow rate. Thus, the first pressure may be measured during one arterial pumping cycle, and the second pressure may be measured during another arterial pumping cycle.

If the dialysis machine 1 is of a single needle configuration using two pumps, the first and second pressures should be measured at two different arterial pump speeds. In the same way as when using clamps, the arterial pressure sensor 6 should be used.

Similarly, if the prescription includes a prescribed flow rate and the detected needle gauge, i.e. needle diameter, is unsuitable for this prescribed flow rate, an alarm event may be generated. The staff in charge of the dialysis machine may then be prompted by a message on the display to change needle gauge or to lower the flow rate.

For being able to check the needle gauge of the blood access device that is connected to the dialysis machine, a table of reference values may be stored in a memory connected to the calculation unit. These reference values may be empirically determined by measuring pressure differences for a range of different needle gauges or needle diameters. The reference values may be determined by measurements made previously on the respective patient. These reference values may be stored on a patient card specific to each patient. Thereby, deviations in pressure differences arising from properties of the blood vessels of the specific patient may be removed as a possible source of error when checking the needle gauge.

In addition to the needle gauge, the pressure difference determined may arise from other properties of the needle set used, but it primarily arises from a combination of the gauge and the length of the needle or needles. To some extent, the pressure difference may arise from the length of the tube section connected to the needle or needles, or the diameter of the tube section. Thus, if one of these factors may be varied by choice of needle or tubing from an available range of needles and tubings, and the other factors are kept constant, the pressure difference may be used to determine an incorrect choice of needle or tubing. As an example, if the operator of the dialysis machine has a range of needles of different gauges to chose from, all of them of the same length, and if only one type of tubing is available, the pressure difference may be used for determining an incorrect needle gauge. If, on the other hand, the operator may choose from a range of needles of different gauges and different lengths and a range of different tubings for the connection of the needles, the pressure difference may only be used for determining if there is something wrong in the extracorporeal line set, i.e. in the combination of needle and tubing. If the determined pressure difference indicates that an incorrect needle set has been connected, the operator may be prompted to check the needle set. The detecting device 20 may be trained to recognize a number of different combinations of needles and tubings by making test measurements and storing the determined pressure difference in the memory. When the detecting device 20 alerts the operator that the combination of the extracorporeal line set connected to the dialysis machine 1 is incorrect, the operator may check the needle set, and, if the needle set is actually a correct combination, but not previously known by the detecting device, the operator may instruct the detecting device to store the data for this new configuration of the extracorporeal line set.

In a dialysis machine including only one pressure sensor, the methods described above may still be used, but then the first pressure and the second pressure will not be measured in different portions of the flow path 2. Instead the two pressures will be measured at one and the same location but at different points in time and at different flow rates or flows in reversed directions.

The method of the invention may be implemented as a computer program. When run on a computer, which may be the calculation unit discussed above, software instructions in the computer program perform the method for detecting a property of a blood access device in the apparatus for extracorporeal blood treatment. The computer program may be stored on a computer-readable medium.

The skilled person realises that a number of modifications of the embodiments described herein are possible without departing from the scope of the invention, which is defined in the appended claims.

For instance, the dialysis machine need not be a hemodialysis machine, but could be a hemodiafiltration machine. Further, the invention is not limited to dialysis, but may be used in any apparatus for extracorporeal blood treatment, such as hemofiltration, hemoadsorption or liver treatment.

In the embodiment described above, the blood access device is a single needle or double needles. Instead of needles, catheters could be used.

The method has here been described at start-up of the dialysis machine, but it may be executed at any time during the treatment. Naturally, for some properties of the extracorporeal line set, such as combination of operation mode and needle configuration, it is important to know as early as possible if there is a mismatch.

The detecting device may, as in the embodiment described above, be an integrated part of the dialysis machine. In such case, the pressure sensors of the detecting device may also used for other purposes in the dialysis machine. Alternatively, the detecting device may be a separate unit, that may be connected to, or built into, a dialysis machine. In such case, the pressure sensors of the detecting device could be either separate pressure sensors integrated in the separate detecting device, or pressure sensors already included in the dialysis machine for other purposes.

1.-21. (canceled)

22. A method for detecting a property of an extracorporeal line set in an apparatus for extracorporeal blood treatment, said extracorporeal line set being arranged to be connected to a patient, said method comprising:
measuring a first pressure in a flow path in said apparatus,
measuring a second pressure in said flow path in said apparatus;
comparing said first pressure to the second pressure, and
determining a detected extracorporeal line set property of said extracorporeal line set based on at least said comparison, wherein said detected extracorporeal line set property is at least one of:
a configuration of a blood access device in said extracorporeal circuit, said configuration being a single access or a double access, and
a blood tubing set property of a blood tubing set in said extracorporeal circuit, said blood tubing set property being any of a single needle single pump set, a single needle double pump set or a double needle set.

23. The method of claim 22, wherein said comparing includes calculating a pressure difference based on the comparison of the first pressure to the second pressure.

24. The method of claim 22, wherein said comparing comprises comparing said pressure difference to a reference value, and said determining comprises determining a detected extracorporeal line set property of said extracorporeal line set based on a result of said comparison to the reference value.

25. The method of 22, wherein said first pressure is measured in a first portion of said flow path and said second pressure is measured in a second portion of said flow path different from said first portion.

26. A method as claimed in claim 25, wherein said first portion of the flow path is connectable to a venous access of a patient undergoing the extracorporeal blood treatment, and said second portion of the flow path is connectable to an arterial access of the patient.

27. The method of claim 22, wherein said first pressure is measured at a first point of time, and said second pressure is measured at a second point of time different from said first point of time.

28. The method of claim 22, further comprising measuring a third pressure, wherein the blood tubing set is determined to be: (a) a single needle single pump set if the first pressure is equal to the second pressure and the third pressure increases slowly; (b) a double needle set if the first pressure is not equal to the second pressure and the third pressure increases rapidly.

29. The method of claim 22, wherein the blood tubing set is determined to be a single needle double pump set if the first pressure initially decreases and when the first pressure has stabilized the first pressure is equal to the second pressure, or the first pressure is initially constant and not equal to the second pressure.

30. The method of claim 22 further comprising:
setting a predetermined extracorporeal line set property,
comparing said detected extracorporeal line set property with said predetermined extracorporeal line set property, and
if said detected extracorporeal line set property is not consistent with said predetermined extracorporeal line set property, generating an alarm event.

31. The method of claim 22, wherein the apparatus for extracorporeal blood treatment is a dialysis machine arranged for use in a single needle mode and a double needle mode.

32. A detection device for detecting a property of an extracorporeal line set in an apparatus for extracorporeal blood treatment, said detection device comprising:
a pressure sensing arrangement and a calculation unit, wherein said pressure sensing arrangement is arranged to measure a first pressure in a flow path in said apparatus and a second pressure in the flow path in said apparatus, and
said calculation unit is arranged to compare the first pressure to the second pressure, and determine an extracorporeal line set property of said extracorporeal line set based on said comparison, wherein said detected extracorporeal line set property is at least one of:
a configuration of a blood access device in said extracorporeal circuit, said configuration being a single access or a double access, and
a blood tubing set property of a blood tubing set in said extracorporeal circuit wherein said blood tubing set property being any of a single needle single pump set, a single needle double pump set or a double needle set.

33. The detection device of claim 32, wherein said calculation unit is arranged to calculate a pressure difference based on said first and second pressures.

34. The detection device of claim 33, wherein said calculation unit is arranged to compare said pressure difference to a reference value, and to determine a detected extracorporeal line set property of said extra-corporeal line set based on a result of said comparison.

35. The detection device of claim 32, wherein said pressure sensing arrangement comprises two pressure sensors, a first of which is arranged to sense the first pressure in a first portion of said flow path and a second of which is arranged to sense the second pressure in a second portion of said flow path.

36. The detection device of claim 35, wherein said first portion of the flow path is connectable to a venous access of a patient, and wherein said second portion of the flow path is connectable to an arterial access of the patient.

37. The detection device of claim 32, wherein said pressure sensing arrangement comprises at least one pressure sensor, said pressure sensor being arranged to sense a first pressure at a first point of time and a second pressure at a second point of time.

38. The detection device of claim 32, wherein the pressure sensing arrangement is arranged to measure a third pressure in the flow path in said apparatus, and wherein the calculation unit is arranged to determine, based on said pressure comparison and said third pressure, that the blood tubing set property of the blood tubing set in said extracorporeal line set is a single needle single pump set if the first pressure is equal to the second pressure and the third pressure increases slowly, and a double needle set if the first pressure is not equal to the second pressure and the third pressure increases rapidly.

39. The detection device of claim 32, wherein the calculation unit is arranged to determine, based on said pressure measurement, that the blood tubing set property of the blood tubing set in said extracorporeal line set is a single needle double pump set if: (a) the first pressure initially decreases, (b) while the first pressure is stable the first pressure is equal to the second pressure, and (b) the first pressure is initially constant and not equal to the second pressure.

40. The detection device of claim 32, further comprising an alarm event generator, said alarm event generator being arranged to generate an alarm event if said detected extracorporeal line set property is not equal to a predetermined extracorporeal line set property.

41. The detection device of claim 32 wherein, said apparatus includes a dialysis machine arranged for use in a single needle mode and a double needle mode.

42. An extracorporeal blood treatment assembly comprising:
an extracorporeal line set including a flow passage;
a first pressure sensor adapted to sense a first pressure in the flow passage at a first location along the passage, and a second pressure sensor adapted to sense a second pressure in the flow passage at a second location along the passage displaced from the first position;
a blood treatment monitor including a mount to receive the extracorporeal line set, processor and a non-transitory memory, wherein the memory stores instructions which are processed by the processor to cause the apparatus to:
receive the pressure data generated by the first and second pressure sensors, and
compare the received pressure data from the first and second pressure sensors, and based on the comparison determine whether the extracorporeal line set is configured for a single access or a double access.

43. The extracorporeal blood treatment assembly of claim 42 wherein the extracorporeal line set includes a blood tubing set and the instructions cause the apparatus to determine a configuration of the blood tubing wherein the configurations include: a single needle single pump set, a single needle double pump set or a double needle set.

44. A non-transitory computer readable medium storing instructions to be executed by a processor to detect a property of an extracorporeal line set in an apparatus for extracorporeal blood treatment, said instructions when executed by the processor cause the apparatus to:
sense a first pressure and a second pressure in a flow path of the extracorporeal line set;
compare said first and second pressures, and
based on the comparison, determine which of a single access configuration and a double access configuration corresponds to the extracorporeal line set.

45. The non-transitory computer readable medium of claim 44 wherein the instructions further cause he apparatus to determine which pump set configuration corresponds to the extracorporeal line set, where the pump set configurations include a single needle single pump set, a single needle double pump set and a double needle pump set the extracorporeal line set is configured for connection.
